(19) United States
(12) Patent Application Publication
(10) Pub. No.: US 2013/0131805 A1
(43) Pub. Date: May 23, 2013

(54) ORTHOPEDIC IMPLANT

(71) Applicants: Marc Hendriks, Brunssum (NL); Detlef Olaf Alexander Schumann, Aachen (DE); Eva Wisse, Eindhoven (NL)

(72) Inventors: Marc Hendriks, Brunssum (NL); Detlef Olaf Alexander Schumann, Aachen (DE); Eva Wisse, Eindhoven (NL)

(21) Appl. No.: 13/578,030

(22) PCT Filed: Feb. 9, 2011
(86) PCT No.: PCT/EP2011/051875

§ 371 Date: Oct. 31, 2012

(30) Foreign Application Priority Data
Feb. 9, 2010 (EP) 10153031.9

(51) Int. Cl. A61F 2/28; A61F 2/44; A61F 2/38

(52) U.S. Cl. CPC A61F 2/28; A61F 2/38; A61F 2/44
USPC 623/17.11; 623/23.61; 623/20.14; 264/241

(57) ABSTRACT

The invention is directed to an orthopedic implant comprising different distinct sections, wherein each section comprises a different polymeric material and the polymeric materials are at the contact surfaces of the sections attached to each other by chemical bonds and/or physical interaction and to a method for the production of an orthopedic implant wherein multi-component molding is used.

The invention is directed to an orthopedic implant, a method for the production of an orthopedic implant and a method for treating the knee or the spine of a mammal by inserting an orthopedic implant into an area of the knee or spine that contains a degenerated, damaged or missing meniscus or spinal disc.

Orthopedic surgery is the branch of surgery concerned with conditions involving the musculoskeletal system. The musculoskeletal system provides for form, stability, and movement of the body. It is made up of the body's bones (the skeleton), muscles, cartilage, tendons, ligaments, joints, and other connective tissue (the tissue that supports and binds tissues and organs together). The musculoskeletal system's primary functions include supporting the body, allowing motion, and protecting vital organs.

The joints and muscoskeletal tissues of the human body are subject to traumatic injury and disease and degenerative processes that over a period of time can lead to the deterioration or failure of the joint causing severe pain or immobility. Generally, the ability of a joint to provide pain free articulation and carry load is dependent upon the presence of healthy bone, cartilage and associated musculoskeletal tissues that provide a stable joint.

In connection with the present invention orthopedic surgery is concerned with maintaining the motion in the various joints of the human body. Examples of orthopedic implants are a meniscus implant or a spinal disc implant.

A meniscus is a crescent-shaped fibrocartilaginous structure that, in contrast to articular disks, only partly divides a joint cavity. In humans it is present in the knee, acromioclavicular, sternoclavicular, and temporomandibular joints. It usually refers to either of two specific parts of cartilage of the knee: the lateral and medial menisci. Both are cartilaginous tissues that provide structural integrity to the knee when it undergoes tension and torsion. The menisci in the knee are often damaged during sports and are then repaired or replaced by surgeons.

Surgical intervention may be required depending on the location of the damage and a repair may be possible. In the outer third of the meniscus, an adequate blood supply exists and a repair will likely heal. Usually younger patients are more resilient and respond well to this treatment, while older patients do not have a favorable outcome after a repair.

The meniscus has fewer vessels and blood flow towards the unattached, thin interior edge. In the majority of cases, the damage is far away from the meniscus' blood supply, and a repair is unlikely to heal. In these cases surgery allows for a partial meniscectomy, removing the torn tissue and allowing the knee to function with some of the meniscus missing. In situations where the meniscus is damaged beyond repair or partial removal, a total meniscectomy is performed. Both options lead to an increased risk of osteoarthritis (with loss of cartilage) and eventual total knee replacement. In some cases, a partial artificial meniscus replacement or total meniscus replacement, by using an allograft, is done to prevent this. An allograft replacement is still a rare procedure and many questions surrounding its use remain.

Meniscus implants are known and are for instance described in U.S.2008086210 and U.S.2009259312. In the last patent publication a meniscus implant is described comprising a central and an outer portion. The implant contains only one type of polymeric material for the central and the outer portion, which is for the outer portion reinforced with fibres. This has the disadvantage that the meniscus implant has the same mechanical properties over its total load-bearing surface.

Spinal discs or intervertebral discs lie between adjacent vertebrae in the spine. Each disc forms a cartilaginous joint to allow slight movement of the vertebrae, and acts as a ligament to hold the vertebrae together. Discs consist of an annular wall (annulus fibrosus) which surrounds the central nucleus (nucleus pulposus). Spinal disc degeneration, characterized by features such as loss of fluid, annular tears and myxomatous changes can result in discogenic pain and/or disc bulging or herniation of the nucleus in which the disc protrudes into the intervertebral foramen comprising spinal verves resulting in back pain and/or sciatica. This condition is more commonly referred to as a “slipped” disc.

To alleviate the condition described above, the damaged spinal disc may be surgically removed from the spine and the two adjacent vertebrae either side of the damaged disc fused together (arthrodesis). Although this technique successfully eliminates the symptoms of pain and discomfort and improves joint stability, it results in a total loss of movement of the fused vertebral joint and increases the stress placed on the adjacent joints leading to collateral damage of these joints and associated soft tissues.

A more desired solution is to replace or repair the damaged spinal disc with an artificial implant that preserves pain free movement of the vertebrae and which mimics the motion and function of the healthy spine.

Spinal disc implants are known and are for instance described in U.S. 2004/0059417. The described spinal implants contain an outer ‘envelope’ which is filled with a curable material in the core. The polymeric materials of the envelope and the core are chemically completely different and do not have any chemical or physical interaction with each other.

The aim of the invention is to provide an orthopedic implant, preferably a cartilage-replacing orthopedic implant, which is chondroprotective and resembles the anatomical shape of, for instance, the spinal disc or the meniscus and which is divided into different distinct sections that have a similar anatomy and mechanical properties as the original cartilage body part.

The aim of the invention is characterized by an orthopedic implant comprising different distinct sections, wherein each section comprises a different polymeric material and the polymeric materials are at the contact surfaces of the sections attached to each other by chemical bonds and/or physical interaction.

This has the advantage that the properties of the polymeric materials can be chosen in such a way that the mechanical properties of the polymeric materials match as good as possible with the mechanical properties of the original cartilage body part. This prevents damage to or subsidence into the tissue that is in contact with the orthopedic implant, improves the lifetime of the implant and gives more comfort to the patient that receives the implant.

The polymeric material used in the orthopedic implant can be a homopolymer, a copolymer, a block copolymer and a random copolymer. The polymer can be selected from, for instance, polyolefins, polyethers, polyesters, polyamides, polystyrenes, polyurethanes, polyacrylates, polysiloxanes and elastomers.

The orthopedic implant can, in each section, contain one or more polymeric materials. The polymeric material in each section is different, with the provision that at the contact surfaces of the sections chemical and/or physical interaction can take place in such a way that the distinct sections are firmly bonded to each other at the contact surfaces.

Different polymeric materials are characterized in that the polymeric materials have different mechanical properties, but are, at the same time, chemically closely related, so that chemical bonds and/or mutual mixing can occur at the contact surfaces. Different mechanical properties of the polymeric materials can be obtained by using polymeric materials with a different chemical composition, but also by using different processing techniques to produce the different distinct sections of the orthopedic implant. Examples of processing techniques are foaming, fused deposition modeling and laser sintering.

The polymeric material is preferably a block copolymer. Block copolymers are polymers comprising hard and soft polymer blocks.

The hard block in the block copolymer comprises a rigid polymer block with a melting temperature (Tm) or a glass transition temperature (Tg) higher than 35° C. The soft block in the block copolymer comprises a flexible, amorphous polymer phase with a Tg lower than 35° C., preferably lower than 0° C. The Tm and Tg were determined on a dry sample.

The block copolymers, used according to the invention, comprise, for example, blends of hard phase polymers with soft phase polymers and block copolymers. The hard and the soft phase can comprise one polymer type, but can also be composed of a mixture of two or more of the above-mentioned polymeric materials.

Mutual mixing and/or chemical bonding of the block copolymers at the contact surfaces of the different distinct sections of the orthopedic implant is very good possible when the hard block and/or the soft block of the different block copolymers used in the different sections of the orthopedic implant have chemical similarity. For instance, when the hard blocks of the block copolymers in the different sections are both polyesters or the soft blocks are both polysiloxanes.

According to one embodiment of the invention the polymeric material can be chosen from a thermoplastic elastomer block copolymers (TPE) comprising a hard block and a soft block, wherein the hard block comprises a polymer chosen from the group consisting of polyester, polyamide, polystyrene, polyacrylate and polyolefin and the soft block comprises a polymer chosen from the group consisting of polyether, polyester, polyacrylate, polyolefin and polysiloxane.

Examples of TPE block-copolymers are block-copolyesterester, block-copolyetherester, block-copolycarbonateester, block-copolysiloxaneester, block-copolyesteramide, block-copolymer containing polybutylene terephthalate (PBT) hard blocks and poly(oxytetramethylene) soft blocks, block-copolymer containing polystyrene hard blocks and ethylene butadiene soft blocks (SEBS), polyurethane comprising polybutylene terephthalate (PBT) hard blocks and polycarbamate soft blocks.

The hard blocks in the thermoplastic elastomer consist of a rigid polymer, as described above, with a Tm or Tg higher than 35° C. In principle the different polymers as described above can be used as the hard blocks. Here and in the rest of the description a polycarbonate or a polycarbamate is understood to be a polyester.

Also copolymers of esters, amides, styrenes, acrylates and olefins can be used as the hard polymer block as long as the Tm or Tg of the hard polymer block is higher than 35° C.

According to another embodiment of the invention the polymeric material can be chosen from polyurethanes. The term polyurethane encompasses a family of polymers that usually includes three principle components. These are a macroglycol, a diisocyanate and a chain extender. They are generally classified as polyurethanes in as much as the backbone thereof includes urethane groups and often also urea groups, which groups are recurring units within the polymer backbone.

With particular reference to the macroglycol component of polyurethanes in general, three primary families of macroglycols are available commercially at the present time. These are the polyester glycols, the polyether glycols and the polycarbonate glycols. The polyester glycols are by far the most widely used macroglycols for polyurethanes at the present time. Polyether urethanes have had some success and are fairly widely used in medical applications. Polycarbonate urethanes are typically more expensive and difficult to process and currently are not in wide use.

Block polyurethane copolymers comprise hard and soft polymer blocks. The hard blocks of the copolymer of the invention may preferably have a molecular weight of about 160 to 10,000, and more preferably about 200 to 2,000. The molecular weight of the soft segment is typically about 200 to 1,000,000 and preferably about 400 to 9000.

Generally known block polyurethane copolymers and methods to prepare these copolymers are described in, for instance, U.S. Pat. No. 4,739,013, U.S. Pat. No. 4,810,749, U.S. Pat. No. 5,133,742 and U.S. Pat. No. 5,229,431.

The biostability of the polyurethane block copolymers in the human body is proven and the polyurethane block copolymers can be chosen in such a way that the mechanical properties of the orthopedic implants resemble the mechanical properties of the original cartilage body part. Therefore, the orthopedic implants preferably contain polyurethane block copolymers.

The polymeric materials that are used may contain one or more additives such as stabilizers, anti-oxidants, colorants, fillers, binders, fibers, meshes, substances providing radio-opacity, surface active agents, foaming agents, processing aids, plasticizers, biostatic/biocidal agents, and any other known agents which are described in Rubber World Magazine Blue Book, and in Gaether et al., Plastics Additives Handbook, (Hanser 1990). Suitable examples of fillers, e.g. radio-opaque fillers and binders are described in U.S. Pat. No. 6,808,585B2 in columns 9-10, which is herein incorporated by reference. Orthopedic implants according to the invention can be produced in radiopaque versions for easy visualization of implant under X-ray. This can be accomplished by one skilled in the art of polymeric fillers and biocompatible materials. For example, barium sulfate, zirconium dioxide, hydroxyapatite, tricalcium phosphate, and other substances which impart radiopacity are described in U.S. Pat. No. 6,808,585 and U.S. Pat. No. 7,044,972 and incorporated here by reference.

The orthopedic implants according to the invention have chondroprotective properties because the polymeric materials that are used to prepare the implants have mechanical properties that resemble the mechanical properties of the original cartilage body part that is replaced. The chondroprotective properties of the implant can be enhanced when the implant comprises components that provide lubricity to the orthopedic implant. This can, for instance, be achieved by surface modification. Surface modification can be performed by using, for instance, polymer compositions as described in WO 95/26993, WO 04/044012 and WO 07/142683.

To fixate the orthopedic implant during implantation it is preferred that at least one suture is incorporated into the implant. This can be achieved by molding in a fiber or thread that can be used as a suture during implantation of the orthopedic implant.

The orthopedic implants can be prepared in many different ways. The distinct sections of the polymeric material can be produced by any known method to shape such polymeric materials. Known techniques include (co-)injection molding, (co-) extrusion molding, blow molding, injection overmolding, MuCell® microcellular foam injection molding, co-extrusion of plates, or creating injection molded foams by decomposing additives like citric acid.

Other processes that can be used are rapid prototyping processes, such as selective layered sintering and fused deposition modeling.

In the orthopedic implants containing more than one polymeric material the different sections made from different polymeric materials can be combined by for instance gluing, welding or molding.

Preferably, multi component molding is used for the preparation of the orthopedic implants according to the invention.

Multi component molding is also called “Two-Shot” or “Multi-Shot” Injection Molding. This is a technology that combines two or more materials in a single mold. Multi component molding makes it possible to produce designs comprising hard and soft parts, or parts with different properties. There are various processes that can be used:

Multi-Shot Over-Molding is the process of molding one plastic over another in one mold. The process is very accurate since the part never leaves the mold. The adhesion of the different materials is superior as the substrate is still hot when the over-molding takes place. Good adhesion prevents separation of the different sections in the orthopedic implant, which may lead to a number of complications including migration, blood vessel and/or nerve damage from the migrated implant.

Multi-Process molding is a method of molding different parts made from different materials in the same mold. This can be especially useful when two parts are used together such as a welded assembly.

In-Mold Assembly is similar to Multi-Process molding, only utilizing mechanisms within the mold to assemble the different parts (when geometry allows) and produce an assembled unit each cycle.

Sandwich Molding, sometimes called “Co-Injection”, is a process where one material is injected through the liquid melt of another material forming a core material with a skin of the other material on the outside.

After molding the orthopedic implant the implant can be processed into its final shape by, for instance, machining or cutting by using mechanical means or by using a fluid jet or an electron beam.

The invention is also directed to orthopedic implants produced by the above-described methods. The orthopedic implant preferably is a meniscus implant or a spinal implant comprising an annular wall and a central nucleus, also referred to as a spinal disk.

Further the invention is directed to a method for treating the knee of a mammal which comprises inserting an orthopedic implant into an area of the knee that contains a degenerated, damaged or missing meniscus. Preferably the orthopedic implant is fixated in the knee with a suture.

In a further embodiment the invention is directed to a method for treating the spine of a mammal which comprises inserting an orthopedic implant into an area of the spine that contains a degenerated, damaged or missing spinal disc.

1. Orthopedic implant comprising different distinct sections, wherein each section comprises a different polymeric material and the polymeric materials are at the contact surfaces of the sections attached to each other by chemical bonds and/or physical interaction.

2. Orthopedic implant according to claim 1, wherein the polymeric material is a block copolymer comprising hard and soft polymer blocks.

3. Orthopedic implant according to claim 1, wherein the polymeric materials of two adjacent distinct sections have chemical similarity in the hard block and/or the soft block present in the polymeric material.

4. Orthopedic implant according to claim 1, wherein the polymeric material is a polyurethane block copolymer.

5. Orthopedic implant according to claim 1, wherein the polymeric material used in the distinct sections on the outside of the orthopedic implant comprise components that provide lubricity to the orthopedic implant.

6. Orthopedic implant according to claim 1, wherein at least one suture is incorporated in the implant.

7. Method for the production of an orthopedic implant according to claim 1, wherein multi-component molding is used to produce at least a part of the orthopedic implant.

8. Orthopedic implant produced by the method according to claim 7.

9. Meniscus implant produced by the method according to claim 7.

10. Spinal implant comprising an annular wall and a central nucleus produced by the method according to claim 7.

11. Method for treating the knee of a mammal which comprises inserting an orthopedic implant according to claim 1 into an area of the knee that contains a degenerated, damaged or missing meniscus.

12. Method according to claim 11, wherein the orthopedic implant is fixed in the knee with a suture.

13. Method for treating the spine of a mammal which comprises inserting an orthopedic implant according to claim 1 into an area of the spine that contains a degenerated, damaged or missing spinal disc.
